(19) United States
(12) Patent Application Publication
(10) Pub. No.: US 2020/0258494 A1
(43) Pub. Date: Aug. 13, 2020

(54) ACOUSTIC LENS, ULTRASONIC PROBE, AND ULTRASONIC DIAGNOSTIC DEVICE

(71) Applicant: OLYMPUS CORPORATION, Tokyo (JP)

(72) Inventor: Kunihisa OBI, Tokyo (JP)

(73) Assignee: OLYMPUS CORPORATION, Tokyo (JP)

(21) Appl. No.: 16/858,784

(22) Filed: Apr. 27, 2020

Related U.S. Application Data
(63) Continuation of application No. PCT/JP2018/034586, filed on Sep. 19, 2018.

(30) Foreign Application Priority Data
Nov. 1, 2017 (JP) 2017-212121

(51) Int. Cl. G10K 11/30; G10K 11/02; A61B 8/00

(52) U.S. Cl. CPC G10K 11/30; A61B 8/4444; G10K 11/02

(57) ABSTRACT

An acoustic lens includes: an elastomer substrate; and a filler which is dispersed in the elastomer substrate and has an average maximum length of 5 μm to 100 μm and an average maximum diameter of 0.01 μm to 1 μm.

### CROSS REFERENCE TO RELATED APPLICATIONS

This application is a continuation application based on a PCT Patent Application No. PCT/JP2018/034586, filed on Sep. 19, 2018, whose priority is claimed on Japanese Patent Application No. 2017-212121, filed on Nov. 1, 2017. The contents of both the PCT Application and the Japanese Application are incorporated herein by reference.

### BACKGROUND

### Technical Field

The present invention relates to an acoustic lens, an ultrasonic probe, and an ultrasonic diagnostic device.

### Background Art

An acoustic lens that focuses an ultrasonic wave is provided in an ultrasonic probe used in an ultrasonic diagnostic device on an outermost surface coming into contact with a subject.

The acoustic lens needs to have acoustic impedance close to that of a subject for the purpose of suppressing reflection of an ultrasonic wave at an interface with the subject.

For example, Japanese Unexamined Patent Application, First Publication No. 2016-107075 discloses an acoustic lens formed of a molded body obtained by dispersing inorganic compound particles having an average primary particle diameter of less than 25 nm and a specific gravity of 2.0 to 10.0 in silicone rubber.

An object of the technology disclosed in Japanese Unexamined Patent Application, First Publication No. 2016-107075 is to significantly improve the hardness and the mechanical strength (tensile rupture strength, tensile rupture elongation, tear strength, and folding fatigue resistance) of a silicone resin while keeping acoustic attenuation low.

However, the tear strength of examples of a resin composition disclosed in Japanese Unexamined Patent Application, First Publication No. 2016-107075 is 17 N/cm to 75 N/cm (1.7 N/mm to 7.5 N/mm). With such a tear strength, there is a concern that an acoustic lens may be damaged in a case where, for example, the acoustic lens comes into contact with or slides on a hard material of other medical devices and the like, or receives a sliding load due to hand-wipe cleaning.

Usage of ultrafine particles having an average primary particle diameter of less than 25 nm is disclosed in Japanese Unexamined Patent Application, First Publication No. 2016-107075. However, the content of such ultrafine particles is limited within a predetermined range for the purpose of obtaining appropriate acoustic characteristics. In the configuration in which ultrafine particles are added, it is impossible to expect to realize the mechanical strength significantly exceed that disclosed in Japanese Unexamined Patent Application, First Publication No. 2016-107075.

Accordingly, there is a strong demand for an acoustic lens which has favorable acoustic characteristics and further improved mechanical durability.

### SUMMARY

The present invention provides an acoustic lens, an ultrasonic probe, and an ultrasonic diagnostic device, all of which have favorable acoustic characteristics and excellent mechanical durability.

An acoustic lens according to a first aspect of the present invention includes: an elastomer substrate; and a filler which is dispersed in the elastomer substrate and has an average maximum length of 5 μm to 100 μm and an average maximum diameter of 0.01 μm to 1 μm.

According to the acoustic lens of a second aspect of the present invention, the filler in the above-described first aspect may include a crystalline inorganic filler.

According to the acoustic lens of a third aspect of the present invention, a material of the filler in the above-described first aspect may include at least one selected from the group consisting of potassium titanate, zinc oxide, and magnesium sulfate.

An ultrasonic probe according to a fourth aspect of the present invention includes an acoustic lens including: an elastomer substrate; and a filler which is dispersed in the elastomer substrate and has an average maximum length of 5 μm to 100 μm and an average maximum diameter of 0.01 μm to 1 μm.

An ultrasonic diagnostic device according to a fifth aspect of the present invention includes an acoustic lens including: an elastomer substrate; and a filler which is dispersed in the elastomer substrate and has an average maximum length of 5 μm to 100 μm and an average maximum diameter of 0.01 μm to 1 μm.

The acoustic lens, the ultrasonic probe, and the ultrasonic diagnostic device of the above-described first to fifth aspects have favorable acoustic characteristics and excellent mechanical durability.

### BRIEF DESCRIPTION OF THE DRAWINGS

FIG. 1 is a schematic front view showing an example of an ultrasonic endoscope as an ultrasonic diagnostic device of a first embodiment of the present invention.

FIG. 2 is a schematic cross-sectional view showing an example of an ultrasonic probe of the first embodiment of the present invention.

FIG. 3 is an enlarged cross-sectional view of an ultrasonic transducer array of the ultrasonic probe of the first embodiment of the present invention.

FIG. 4 is a block diagram showing an example of a configuration of a control system of the ultrasonic endoscope as the ultrasonic diagnostic device of the first embodiment of the present invention.

FIG. 5 is a schematic cross-sectional view of an acoustic lens of the first embodiment of the present invention.

FIG. 6 is a schematic cross-sectional view of an acoustic lens of a modification example of the first embodiment of the present invention.

FIG. 7 is a schematic system configuration diagram showing an example of an ultrasonic diagnostic device as a medical device of a second embodiment of the present invention.

### DETAILED DESCRIPTION OF THE PREFERRED EMBODIMENT

Hereinafter, embodiments of the present invention will be described with reference to the accompanying drawings. In all the drawings, the same or corresponding members will be denoted by the same reference numerals even in cases of different embodiments, and the common description will not be repeated.

### First Embodiment

Hereinafter, an acoustic lens, an ultrasonic probe, and an ultrasonic diagnostic device of a first embodiment of the present invention will be described.

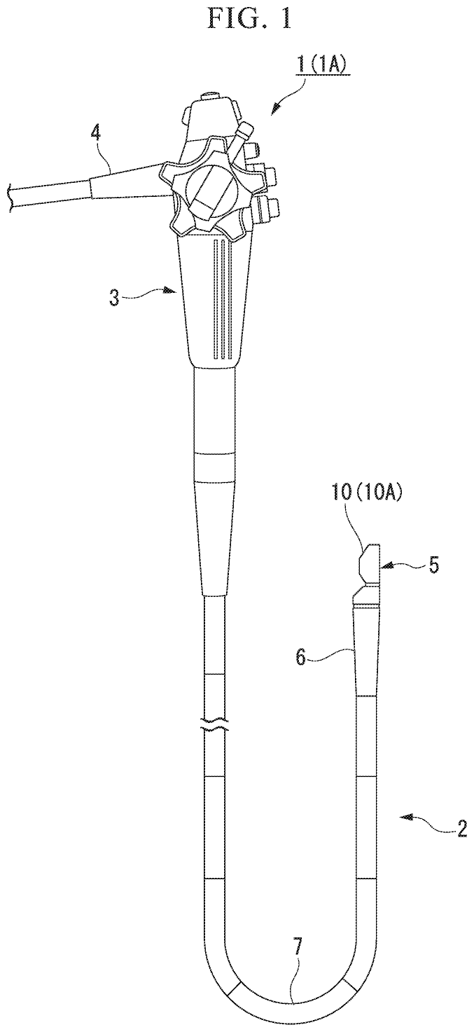

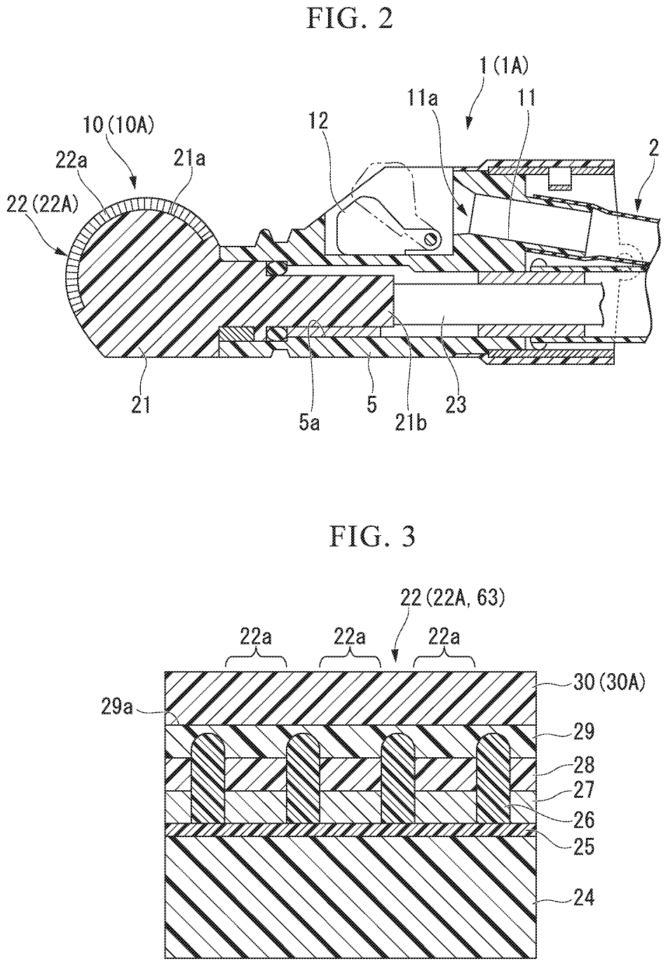

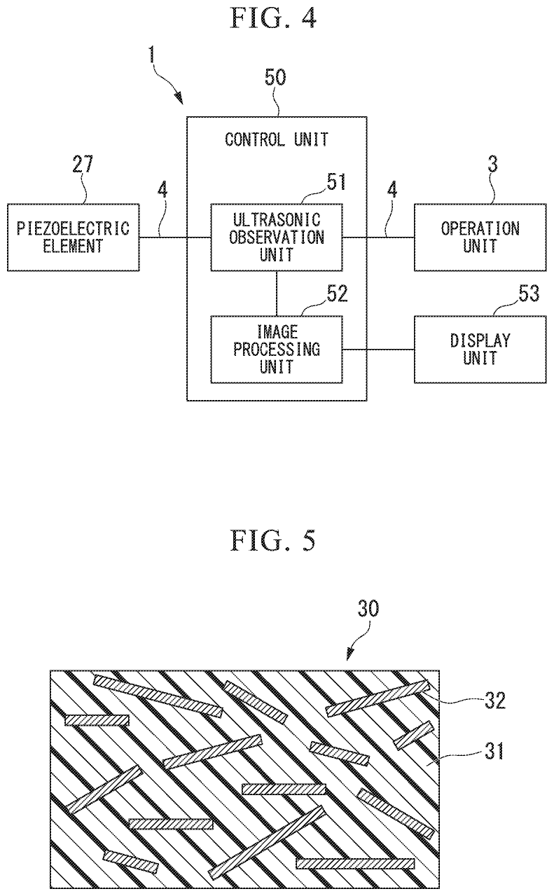

FIG. 1 is a schematic front view showing an example of an ultrasonic endoscope as an ultrasonic diagnostic device of a first embodiment of the present invention. FIG. 2 is a schematic cross-sectional view showing an example of an ultrasonic probe of the first embodiment of the present invention. FIG. 3 is an enlarged cross-sectional view of an ultrasonic transducer array of the ultrasonic probe of the first embodiment of the present invention. FIG. 4 is a block diagram showing an example of a configuration of a control system of the ultrasonic endoscope as the ultrasonic diagnostic device of the first embodiment of the present invention.

An ultrasonic endoscope 1 (ultrasonic diagnostic device) of the present embodiment shown in FIG. 1 is a medical endoscope used by being inserted into the body of a patient. The ultrasonic endoscope 1 can acquire an ultrasonic image of a subject by applying an ultrasonic wave to the subject.

The ultrasonic endoscope 1 includes an insertion unit 2, an operation unit 3, and a universal cord 4. The shape of the insertion unit 2 is elongated. The insertion unit 2 is inserted into the body of a patient. The operation unit 3 is connected to a proximal end of the insertion unit 2. The universal cord 4 extends from the operation unit 3.

The insertion unit 2 is formed in a tubular shape which has flexibility so as to be inserted into the body of a patient. A distal end hard portion 5, a curved portion 6, and a flexible tube portion 7 are provided in the insertion unit 2 in this order from a distal side in an insertion direction. The curved portion 6 is freely curved. The shape of the flexible tube portion 7 has a small diameter and is long. The flexible tube portion 7 has flexibility.

Although not shown in FIG. 1, long built-in objects such as a treatment tool channel, a light guide fiber, an image transmission cable, and an operation wire are inserted into the insertion unit 2, for example.

The distal end hard portion 5 is formed of a hard resin-molded product. The shape of the distal end hard portion 5 is substantially cylindrical. The distal end hard portion 5 includes an end effector of the ultrasonic endoscope 1 which functions as a manipulator of the ultrasonic endoscope 1. An ultrasonic probe 10 is provided at a distal portion of the distal end hard portion 5. Although not shown in the drawing, an imaging element, an illumination optical system, an imaging optical system, and the like are provided in the distal end hard portion 5 for the purpose of acquiring an image of a subject. The imaging element is composed, for example, of a CCD. The illumination optical system emits illumination light. The imaging optical system includes an appropriate lens.

As shown in FIG. 2, a treatment tool channel 11 extends in the distal end hard portion 5. A treatment tool or the like is inserted through the treatment tool channel 11. A treatment tool or the like can be extended to the outside through an opening portion 11a of the treatment tool channel 11. A raising base 12 is provided in front of the opening portion 11a. The raising base 12 can change the direction of a treatment tool or the like extending from the opening portion 11a to the outside.

An ultrasonic probe mounting hole 5a is formed next to the raising base 12 at a distal end of the distal end hard portion 5. The ultrasonic probe 10 is fixed to the ultrasonic probe mounting hole 5a.

The ultrasonic probe 10 includes a main body portion 21 and an ultrasonic transducer array 22. The ultrasonic transducer array 22 is provided on the surface of the main body portion 21.

An array arrangement surface 21a is formed at a distal end of the main body portion 21. The ultrasonic transducer array 22 to be described below is disposed on the array arrangement surface 21a. In the present embodiment, the array arrangement surface 21a has a cylindrical surface shape.

A fixing unit 21b is formed at a proximal end of the main body portion 21. The fixing unit 21b is connected and fixed to the distal end hard portion 5 in a state of being inserted into the ultrasonic probe mounting hole 5a.

The ultrasonic transducer array 22 is configured such that a plurality of ultrasonic transducers 22a are arranged on the array arrangement surface 21a.

As shown in FIG. 3, a backing material 24, a piezoelectric element 27, a first acoustic matching layer 28, a second acoustic matching layer 29, and an acoustic lens 30 are stacked in each of the ultrasonic transducers 22a in this order. Each ultrasonic transducer 22a is partitioned by a partition wall 26 into a rectangular shape. The partition wall 26 penetrates the piezoelectric element 27 and the first acoustic matching layer 28 in a layer thickness direction.

The ultrasonic transducers 22a partitioned by the partition wall 26 can execute transmission and reception of ultrasonic waves. Each ultrasonic transducer 22a generates an image signal. The image signal generated by each ultrasonic transducer 22a is an image signal of a unit pixel in an ultrasonic image acquired by the ultrasonic transducer array 22.

The backing material 24 adheres to the array arrangement surface 21a not shown in the drawings through interposition of an appropriate adhesive. The backing material 24 is a member that absorbs vibration toward the array arrangement surface 21a among ultrasonic vibrations generated by the piezoelectric element 27 to be described below.

A resin material having appropriate vibration absorption characteristics is used as the material of the backing material 24.

In a case where a voltage is applied by an electrode not shown in the drawings, the piezoelectric element 27 generates ultrasonic vibration. Furthermore, in a case where an ultrasonic wave reflected from the outside of the ultrasonic probe 10 is incident, the piezoelectric element 27 converts the ultrasonic vibration into an electrical signal.

Wiring, not shown in the drawings, of each ultrasonic transducer 22a is electrically connected to the piezoelectric element 27. Each wiring extends from the fixing unit 21b (refer to FIG. 2) in a state of being put together in a cable 23 shown in FIG. 2.

The cable 23 extending from the fixing unit 21b extends to the operation unit 3 to be described below via the inside of the ultrasonic probe mounting hole 5a and the inside of the insertion unit 2.

As shown in FIG. 3, an insulating layer 25 is provided between each piezoelectric element 27 and each partition wall 26. The insulating layer 25 electrically insulates the piezoelectric elements 27 and the backing material 24 from each other. The insulating layer 25 is formed of an organic substance, for example, an adhesive, which does not contain a conductor.

The first acoustic matching layer 28 and the second acoustic matching layer 29 are layered portions that reduce the difference in acoustic impedance between a subject and the piezoelectric element 27. In a case where the acoustic impedance of the first acoustic matching layer 28 and the acoustic impedance of the second acoustic matching layer 29 are appropriately set according to the acoustic impedance of the subject, reflection of ultrasonic waves from the subject is reduced.

In the present embodiment, the first acoustic matching layer 28 is partitioned into a rectangular shape by each partition wall 26. In contrast, in the present embodiment, the second acoustic matching layer 29 is set to have a layer shape that covers distal ends of each first acoustic matching layer 28 and each partition walls 26.

However, the first acoustic matching layer 28 and the second acoustic matching layer 29 may be replaced with a single acoustic matching layer.

The acoustic lens 30 focuses ultrasonic waves which are generated by the piezoelectric element 27 and propagate through the first acoustic matching layer 28 and the second acoustic matching layer 29, and radiates the ultrasonic waves to the outside. Furthermore, the acoustic lens 30 focuses the ultrasonic waves reflected from the outside and makes the ultrasonic waves incident on each piezoelectric element 27. The acoustic lens 30 is molded into an appropriate shape for the purpose of focusing the ultrasonic waves.

The acoustic lens 30 is disposed on the outermost surface of the ultrasonic probe 10.

The details of the acoustic lens 30 will be described after the description of the ultrasonic endoscope 1 is finished.

As shown in FIG. 1, the curved portion 6 is connected to a proximal end of the distal end hard portion 5. The curved portion 6 is a tubular portion configured so as to be curved for the purpose of changing the direction of the distal end hard portion 5.

The curved portion 6 includes, for example, a plurality of annular joint rings. The plurality of joint rings are rotatably connected to each other. An operation wire is inserted into the plurality of joint rings.

Members such as electrical wiring connected to an imaging element of the distal end hard portion 5 and a light guide fiber extending to an illumination window are accommodated in the curved portion 6, for example.

The above-described members such as the operation wire, the image transmission cable, the light guide fiber, and the cable 23 which is not shown in the drawings are inserted into the flexible tube portion 7 to be described below and extend to the operation unit 3 to be described below.

The curved portion 6 is covered with an outer tube made of a resin.

The flexible tube portion 7 is a tubular portion that connects the curved portion 6 to the operation unit 3 to be described below.

The flexible tube portion 7 includes a resin tube having a lumen. Reinforcing members such as flex and a metal braid may be provided in the resin tube for the purpose of maintaining the circular cross section of the lumen. In the flex, a strip-like member made of metal or a resin is wound in a spiral shape, for example. For example, a metal braid has a net shape.

The operation unit 3 is a device part which an operator uses for the purpose of operating the ultrasonic endoscope 1. An example of the operation using the operation unit 3 includes an operation of pulling an operation wire not shown in the drawings for the purpose of changing the amount of curvature of the curved portion 6. Another example of the operation using the operation unit 3 includes an operation of driving the ultrasonic probe 10 to acquire an ultrasonic image.

The universal cord 4 is connected to a proximal portion of the operation unit 3. The universal cord 4 connects a power supply line, a signal line, and the like necessary for the operation of the ultrasonic endoscope 1 to an external device.

As shown in FIG. 4, the ultrasonic endoscope 1 further includes a control unit 50 and a display unit 53. The display unit 53 displays an image.

The control unit 50 includes an ultrasonic observation unit 51 and an image processing unit 52.

The ultrasonic observation unit 51 is electrically connected to the operation unit 3 and the piezoelectric elements 27 through the universal cord 4. The ultrasonic observation unit 51 transmits and receives an ultrasonic wave based on a control signal from the operation unit 3. The transmission and reception of an ultrasonic wave are executed using the piezoelectric elements 27. Specifically, the ultrasonic observation unit 51 sequentially radiates an ultrasonic wave from each piezoelectric element 27 of each ultrasonic transducer 22a. The ultrasonic observation unit 51 receives reflected wave of the radiated ultrasonic waves via each ultrasonic transducer 22a (refer to FIG. 3). Accordingly, the ultrasonic observation unit 51 acquires echo data.

The image processing unit 52 analyzes the echo data acquired by the ultrasonic observation unit 51. The image processing unit 52 executes image processing for constructing an ultrasonic image based on the analysis results of the echo data.

The display unit 53 is communicably connected to the image processing unit 52. The image signal of the ultrasonic image constructed by the image processing unit 52 is sent to the display unit 53. Accordingly, the ultrasonic image is displayed on the display unit 53.

The device configuration of the control unit 50 includes a computer including a CPU, a memory, an input and output interface, an external storage device, and the like, thereby executing a program for operating the ultrasonic observation unit 51 and the image processing unit 52 as described above.

Next, a detailed configuration of the acoustic lens 30 of the present embodiment will be described.

FIG. 5 is a schematic cross-sectional view of an acoustic lens of the first embodiment of the present invention.

As schematically shown in FIG. 5, the acoustic lens 30 includes an elastomer substrate 31 and a filler 32.

The elastomer substrate 31 is made of a cured product of a resin material. The resin material used for the elastomer substrate 31 has biocompatibility suitable for contact with biological tissue (subject). Acoustic impedance close to that of the subject is obtained from the resin material used for the elastomer substrate 31 by addition of the filler 32 to be described below.

For example, millable type silicone rubber may be used as the elastomer substrate 31. In this case, the millable type silicone rubber may be manufactured by adding a vulcanizing agent to a raw material resin to heat and cure the raw material resin. For example, the raw material resin may be configured to include diorganopolysiloxane as a main component and polysiloxane having a polymerization degree of 3,000 to 10,000.

For example, liquid rubber type silicone rubber may be used as the elastomer substrate 31. In this case, the liquid rubber type silicone rubber may include diorganopolysiloxane as a main component and be manufactured by adding a curing agent to polysiloxane having a low polymerization degree to cure the polysiloxane. There are a condensation reaction type and an addition reaction type in such liquid rubber type silicone rubber. In particular, there is an advantage that no by-products are generated during curing in the case of the addition reaction type.

However, the elastomer substrate 31 is not limited to the above-described kinds and types. The material of the elastomer substrate 31 may be appropriately selected from elastomer materials other than the above-described silicone rubber in consideration of, for example, biocompatibility, sterilization resistance, flexibility, durability, and curability.

The filler 32 is blended in a state of being dispersed in the elastomer substrate 31. The filler 32 has a shape with an average maximum length of 5 μm to 100 μm and an average maximum diameter of 0.01 μm to 1 μm. The filler 32 more preferably has a shape with an average maximum length of 10 μm to 50 μm and an average maximum diameter of 0.1 μm to 0.6 μm.

Here, the “average maximum length” is a value obtained by averaging the maximum external dimensions of a single filler 32 contained in the acoustic lens 30 in a group of fillers 32 contained in the acoustic lens 30.

The “average maximum diameter” is a value obtained by averaging the maximum line diameters of a linear portion of a single filler 32 contained in the acoustic lens 30 in a group of fillers 32 contained in the acoustic lens 30.

The shape of a filler 32 is not particularly limited as long as the average maximum length and the average maximum diameter satisfy the above-described numerical ranges. For example, the shape of the filler 32 may be fibrous. For example, the filler 32 may be a linear body having a linear shape, or may be a curved or bent linear body. For example, the filler 32 may have a shape in which a linear body is branched in a tree shape. For example, the filler 32 may be a rod-like linear body having a substantially constant cross-sectional area, or may be a needle-like linear body having a variable cross-sectional area. For example, the filler 32 may have a hollow tubular shape.

The material of the filler 32 may be an inorganic filler or an organic filler. However, the filler 32 is more preferably an inorganic filler from the viewpoint that a high specific gravity is easily obtained. A carbon nanotube may be used as the material of the filler 32 from the viewpoint that high strength is obtained while the specific gravity is low.

In a case where the filler 32 is an inorganic filler, the filler 32 is more preferably a crystalline material from the viewpoint that the shape in the above-described numerical range is easily produced. In the case of the crystalline material, a linear body having stable maximum length and maximum outer diameter is easily obtained by crystal growth. In the case where the filler 32 is made of a crystalline material, it is particularly preferable to produce the filler by making a single crystal grow. In the case of the crystalline inorganic filler produced by crystal growth, the filler itself has a high strength compared to a filler produced through mechanical processing or the like. As a result, the reinforcing effect of the crystalline inorganic filler is enhanced.

However, the filler 32 may be produced using a non-crystalline material (amorphous material) as long as the shape in the above-described numerical range is obtained. Specifically, a glass filler or the like may be used as the filler 32.

Examples of materials suitable for the filler 32 include potassium titanate, zinc oxide, and magnesium sulfate. For example, the material of the filler 32 may include at least one selected from the group consisting of potassium titanate, zinc oxide, and magnesium sulfate.

In order to manufacture the acoustic lens 30 of the present embodiment, a molding material in which a vulcanizing agent or a curing agent and the filler 32 are added is formed in the elastomer substrate 31 before curing, for example. In the molding material, a vulcanizing agent or a curing agent and the filler 32 are substantially uniformly dispersed and mixed with each other. The formed molding material is cured using a molding die for forming the shape of the acoustic lens 30. The acoustic lens 30 is manufactured by being demolded from the cured product of the molding material.

The action of such an ultrasonic endoscope 1 will be described focusing on the action of the acoustic lens 30.

The acoustic lens 30 contains the filler 32 having an average maximum length of 5 μm to 100 μm and an average maximum diameter of 0.01 μm to 1 μm.

The filler 32 has such a shape having a large aspect ratio. As a result, the filler 32 has a larger specific surface area than a filler with granular bodies. Accordingly, the filler 32 has a larger contact area with the elastomer substrate 31 than that with granular bodies. As a result, the integrity with the elastomer substrate 31 increases.

As a result of the increased integrity with the elastomer substrate 31, the reinforcing effect of the filler 32 against external forces of compression and tension in a longitudinal direction particularly is enhanced.

In contrast, in the filler with granular bodies, the specific surface area increases in a case where the particle diameter of the filler is reduced. As a result, the reinforcing effect is enhanced to some extent compared to a case where the same amount of filler having a larger particle diameter is added. However, the degree of the enhancement in the reinforcing effect is remarkably smaller than that of the filler 32 having a large aspect ratio.

The filler 32 has a remarkably larger aspect ratio than that with granular bodies. As a result, the reinforcing effect of one filler 32 reaches a wider area.

In contrast, in the filler with granular bodies, the representative length decreases as the particle diameter decreases. As a result, a region which the reinforcing effect of one filler reaches becomes narrower.

It is necessary to increase the content of the filler in order to further enhance the reinforcing effect using the filler with granular bodies. In this case, the mixture with the elastomer substrate 31 becomes hard and brittle. As a result, the tear strength or the like particularly tends to decrease. Furthermore, in a case where the content of the filler is too large, the acoustic characteristics easily deviate from an appropriate range.

In particular, in a case where the filler 32 has a shape of a branched linear portion, the branched linear portion has an anchoring effect with respect to the elastomer substrate 31. As a result, the reinforcing effect using the filler 32 is further enhanced.

In this case, the branched linear portion also has an effect of being easily entangled with linear portions of other fillers 32. It is possible to expect enhancement in the reinforcing effect by increasing mutual entanglement of the fillers 32 dispersed in the elastomer substrate 31.

In the acoustic lens 30, the compressive strength and the tensile strength of the filler 32 in the longitudinal direction are particularly improved as described above.

Furthermore, in the acoustic lens 30, the strength against a shear force acting in a direction intersecting the longitudinal direction of the filler 32 is improved, for example. As a result, the progress of microcracks generated in the elastomer substrate 31 during, for example, rupture or tearing is suppressed by the filler 32. In this manner, in the acoustic lens 30, the rupture strength and the tear strength are also improved compared to the case where granular bodies are added.

The filler 32 has a high aspect ratio, but the average maximum length is less than or equal to 100 μm. As a result, the orientation in a case where the filler is mixed with the elastomer substrate 31 before curing is low. Accordingly, the elastomer substrate 31 and the filler 32 before curing can be mixed with each other in a substantially uniformly dispersed state. Accordingly, the filler 32 is substantially not oriented. As a result, the anisotropy of the tensile strength, the rupture strength, and the tear strength of the acoustic lens 30 are suppressed.

The reinforcing effect of such a filler 32 increases as the average maximum length is longer or as the average maximum diameter is larger.

On the other hand, the filler 32 also has an effect of changing the acoustic characteristics of the acoustic lens 30 according to the shape and content of the acoustic lens 30. Specifically, the filler 32 attenuates ultrasonic waves transmitted through the acoustic lens 30 or changes the acoustic impedance of the acoustic lens 30.

The specific gravity, the average maximum length, and the average maximum diameter of the filler 32 are selected for the purpose of making the above-described reinforcing effect and the acoustic characteristics of the acoustic lens 30 appropriate.

For example, the reinforcing effect becomes too small in a case where the average maximum diameter of the filler 32 is less than 0.01 μm (10 nm). The attenuation rate of ultrasonic waves becomes too high in a case where the average maximum diameter of the filler 32 exceeds 1 μm.

For example, a range which the reinforcing effect reaches becomes too small in a case where the average maximum length of the filler 32 is less than 5 μm. As a result, the tear strength of the acoustic lens 30 particularly excessively decreases. The attenuation rate of ultrasonic waves of the acoustic lens 30 becomes too high in a case where the average maximum length of the filler 32 exceeds 100 μm.

For example, it is more preferable that the material with a high specific gravity is selected as a material of the filler 32 for the purpose of adjusting the acoustic impedance of the acoustic lens 30 without much increasing the content. The specific gravity of the filler 32 is more 10preferably 1.3 to 10. The specific gravity of the filler 32 is still more preferably 2 to 6.

For example, potassium titanate (specific gravity of 3.3 to 3.5), zinc oxide (specific gravity of 5.6 to 5.8), and magnesium sulfate (specific gravity of 2.3 to 2.6) are more preferable in terms of the specific gravity.

Potassium titanate, zinc oxide, and magnesium sulfate are crystalline materials. The strength of crystalline materials becomes high in a case where these are produced by crystal growth. Accordingly, in a case where the filler 32 is produced from crystal-grown crystalline materials, the strength of the filler itself becomes high. In this case, a necessary reinforcing effect is obtained even if the average maximum diameter is small. As a result, the decrease in the attenuation rate is suppressed.

In this manner, the acoustic lens 30 contains the above-described filler 32 in the ultrasonic endoscope 1. As a result, the acoustic lens 30 has favorable acoustic characteristics and excellent mechanical durability.

For example, the acoustic lens 30 particularly has an excellent tear strength. Accordingly, the acoustic lens 30 is hardly damaged even if the acoustic lens 30 comes into contact with or slides on a hard material of other medical devices and the like, or receives a sliding load due to hand-wipe cleaning. As a result, the durability life of the acoustic lens 30 is improved. Similarly, the durability of the ultrasonic probe 10 and the ultrasonic endoscope 1 which include the acoustic lens 30 is also improved.

### Modification Example

Next, a modification example of the first embodiment of the present invention will be described.

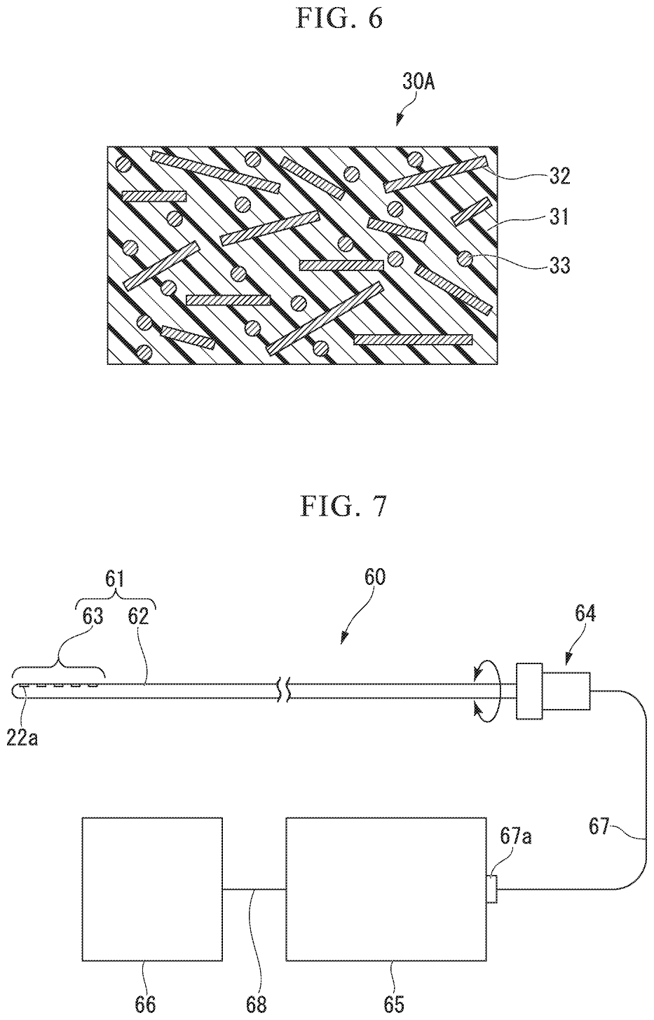

FIG. 6 is a schematic cross-sectional view of an acoustic lens of a modification example of the first embodiment of the present invention.

As shown in FIG. 1, an ultrasonic endoscope 1A (ultrasonic diagnostic device) of the present modification example includes an ultrasonic probe 10A instead of the ultrasonic probe 10 of the ultrasonic endoscope 1 of the above-described first embodiment.

As shown in FIG. 2, the ultrasonic probe 10A includes an ultrasonic transducer array 22A instead of the ultrasonic transducer array 22 of the above-described first embodiment.

Hereinafter, the difference from the above-described first embodiment will be described.

As shown in FIG. 3, the ultrasonic transducer array 22A of the present modification example includes an acoustic lens 30A instead of the acoustic lens 30 of the above-described first embodiment.

As schematically shown in FIG. 6, the acoustic lens 30A further includes a filler 33 in addition to the elastomer substrate 31 and the filler 32.

The filler 33 is made of inorganic particles. The average aspect ratio of the filler 33 is smaller than that of the filler 32.

An appropriate amount of the filler 33 is contained in the acoustic lens 30A together with the filler 32 so as to obtain acoustic characteristics necessary for the acoustic lens 30A.

The filler 33 preferably has a high specific gravity.

In order to prevent the attenuation rate of the acoustic lens 30A from becoming excessively large, the average particle diameter of the filler 33 is more preferably 0.01 μm to 1 μm.

Examples of inorganic fillers suitable as the filler 33 include silica, alumina, boehmite, cerium oxide, boron nitride, aluminum nitride, magnesium oxide, aluminum hydroxide, zinc oxide, tungsten trioxide, zirconia, diamond, silicon nitride, silicon carbide, and sapphire.

As the filler, one kind of substance may be used, or two or more kinds of substances may be used.

According to the acoustic lens 30A of the present modification example, the acoustic lens contains the filler 33 in addition to the filler 32. As a result, the acoustic lens 30A has excellent mechanical durability in the same manner as in the above-described first embodiment.

Furthermore, according to the present modification example, the acoustic characteristics can be appropriately adjusted by blending the filler 32 with the filler 33. For example, in a case where a material with a low specific gravity is used as the filler 32, the acoustic impedance sometimes becomes too low with the content of the filler 32 from which a necessary strength is obtained. In this case, it is possible to adjust the acoustic impedance to an appropriate value by appropriately incorporating the filler 33 having a high specific gravity.

As a result, the acoustic lens 30A of the present modification example has favorable acoustic characteristics and excellent mechanical durability. Accordingly, the durability life of the acoustic lens 30A is improved. Similarly, the durability of the ultrasonic probe 10A and the ultrasonic endoscope 1A which include the acoustic lens 30A is also improved.

### Second Embodiment

Next, an ultrasonic probe and an ultrasonic diagnostic device of a second embodiment of the present invention will be described.

FIG. 7 is a schematic system configuration diagram showing an example of an ultrasonic diagnostic device as a medical device of a second embodiment of the present invention.

As shown in FIG. 7, an ultrasonic diagnostic device 60 of the present embodiment is used in a state of being inserted into the body of a patient via, for example, an endoscope. The ultrasonic diagnostic device 60 can acquire an ultrasonic image of a subject by applying an ultrasonic wave to the subject.

The ultrasonic diagnostic device 60 includes an ultrasonic probe 61, a probe rotating unit 64, a control unit 65, and a monitor 66.

The ultrasonic probe 61 includes a probe main body 62 and an ultrasonic application unit 63. The probe main body 62 is formed of a sheath member made of a resin. The shape of the sheath member is elongated. The sheath member has flexibility. The ultrasonic application unit 63 is provided at a distal portion of the probe main body 62.

The probe main body 62 has an outer diameter capable of being inserted into a treatment tool channel of an endoscope, for example. The probe main body 62 has a length capable of extending from an opening of a distal portion of the treatment tool channel, for example.

The ultrasonic application unit 63 includes the ultrasonic transducers 22a in the same manner as in the above-described first embodiment. A plurality of the ultrasonic transducers 22a in the ultrasonic application unit 63 are arranged in a longitudinal direction of the probe main body 62. The row of the ultrasonic transducers 22a in the present embodiment is not limited to one row. For example, the ultrasonic transducers 22a may have two rows in a state in which the rows are opposed to each other while interposing a central axis of the probe main body 62.

As shown in FIG. 3, the acoustic lens 30 similar to that of the above-described first embodiment is disposed on an outermost surface of the ultrasonic transducers 22a of the ultrasonic application unit 63.

Signal lines not shown in the drawings are respectively connected to the piezoelectric elements 27 of the ultrasonic transducers 22a. Each signal line is inserted into the probe main body 62. Each signal line extends to the probe rotating unit 64 to be described below. The signal lines are collected in a signal cable 67 as shown in FIG. 7. Each signal line extends from the probe rotating unit 64 to the outside. The signal cable 67 is connected to the control unit 65 to be described below through a connector 67a. The ultrasonic application unit 63 and the control unit 65 to be described below are communicably connected to each other through the signal cable 67.

The probe rotating unit 64 is a device part that is provided for the purpose of carrying out a rotary operation of the ultrasonic probe 61.

In the present embodiment, an operator can manually rotate the ultrasonic probe 61 around its central axis with the probe rotating unit 64. A main part of the probe rotating unit 64 is constituted of a plurality of resin members. The plurality of resin members are relatively rotatably assembled with each other.

The control unit 65 controls transmission and reception of ultrasonic waves through each of the piezoelectric elements 27 (refer to FIG. 3). Furthermore, the control unit 65 analyzes echo data, which each of the piezoelectric elements 27 received, and executes image processing such as construction of ultrasonic images. The signal cable 67 is electrically connected to the control unit 65 through the connector 67a.

The same configuration as that of the control unit 50 in the above-described first embodiment is used as the device configuration of the control unit 65.

The monitor 66 displays ultrasonic images and the like constructed by the control unit 65. The monitor 66 is communicably connected to the control unit 65 through a signal cable 68.

The ultrasonic probe 61 of the ultrasonic diagnostic device 60 with such a configuration is used in a state in which, for example, a distal portion of the ultrasonic probe 61 is inserted into the body of a patient through a treatment tool channel or the like of an endoscope. The ultrasonic probe 61 has flexibility. As a result, in a case where the treatment tool channel is curved, the ultrasonic probe is inserted thereinto while being bent along the curvature of the treatment tool channel.

The ultrasonic probe 61 inserted into the body of a patient is rotated by the probe rotating unit 64 for the purpose of bringing the ultrasonic application unit 63 into contact with a subject within the body of the patient.

In a case where the ultrasonic application unit 63 comes into contact with the subject, transmission and reception of ultrasonic waves are executed by each of the piezoelectric elements 27 of the ultrasonic application unit 63. The control unit 65 acquires echo data. The acquired echo data is analyzed by the control unit 65. The control unit 65 constructs ultrasonic images. An image signal of an ultrasonic image is transmitted to the monitor 66. The image signal of the ultrasonic image is displayed on the monitor 66.

When using the ultrasonic probe 61 in this manner, the acoustic lens 30 on the surface of the ultrasonic application unit 63 slides on the treatment tool channel or the subject. Furthermore, the acoustic lens 30 of the ultrasonic probe 61 receives, for example, a sliding load due to hand-wipe cleaning before and after use.

However, the acoustic lens 30 has excellent mechanical durability, and is therefore hardly damaged.

As a result, the durability of the ultrasonic probe 61 and the ultrasonic diagnostic device 60 which include the acoustic lens 30 is also improved.

In the description of each of the above-described embodiments, a case where the acoustic lenses 30 and 30A include the elastomer substrate 31 and the filler 32 has been described as an example. Appropriate additives may be contained in the acoustic lenses 30 and 30A as necessary. The type and the content of the additives contained in the acoustic lenses 30 and 30A are not particularly limited as long as these are within a range where acoustic characteristics required for the acoustic lens 30 can be obtained. For example, additives such as a moistening agent and a coloring material may be added to the acoustic lenses 30 and 30A as long as these are within a range where acoustic characteristics required as the acoustic lenses 30 and 30A can be obtained.

### EXAMPLES

Hereinafter, examples of resin compositions forming an acoustic lens corresponding to the above-described first embodiment and modification example will be described together with a comparative example.

The compositions and evaluation results of the resin compositions of Examples 1 to 5 are shown in Table 1 below together with the composition and an evaluation result of a resin composition of a comparative example.

TABLE 1

| | Elastomer | | Filler | | | | Inorganic particles | | | | |
|---|---|---|---|---|---|---|---|---|---|---|---|
| | substrate | | | Average | Average | | | Average | | Evaluation | |
| | | Parts | | maximum | maximum | Parts | | maximum | Parts | Acoustic | Tear |
| | | by | | length | diameter | by | | diameter | by | impedance | strength |
| | Material | mass | Material | (μm) | (μm) | mass | Material | (μm) | mass | (MRayl) | (N/mm) |
| Example 1 | Silicone | 65 | Potassium | 15 | 0.4 | 35 | — | — | — | 1.30 | 43 |
| | rubber | | titanate | | | | | | | | |
| Example 2 | Silicone | 55 | Zinc oxide | 10 | 0.2 | 45 | — | — | — | 1.40 | 48 |
| | rubber | | | | | | | | | | |
| Example 3 | Silicone | 65 | Potassium | 15 | 0.4 | 30 | Alumina | 0.03 | 5 | 1.30 | 41 |
| | rubber | | titanate | | | | | | | | |
| Example 4 | Silicone | 60 | Magnesium | 15 | 0.5 | 30 | Zirconia | 0.1 | 10 | 1.30 | 41 |
| | rubber | | sulfate | | | | | | | | |
| Example 5 | Silicone | 65 | Potassium | 15 | 0.4 | 30 | — | — | — | 1.30 | 49 |
| | rubber | | Litanate | | | | | | | | |
| | | | Glass | 100 | 1 | 5 | | | | | |
| Comparative | Silicone | 65 | — | — | — | — | Alumina | 1 | 35 | 1.31 | 11 |
| Example | rubber | | | | | | | | | | |

### Example 1

Example 1 is an example of a resin composition forming the acoustic lens 30 of the first embodiment.

In the resin composition of Example 1, 65 parts by mass of silicone rubber was used as the elastomer substrate 31 and 35 parts by mass of a crystalline filler made of potassium titanate was used as the filler 32 as shown in [Table 1].

An addition reaction type liquid rubber composed of two kinds of liquids was used as the silicone rubber. A platinum catalyst was used as a curing agent.

The average maximum length of the filler was 15 μm, and the average maximum diameter thereof was 0.4 μm.

The above-described liquid rubber and the above-described filler were weighed for the purpose of producing evaluation samples of the resin composition of Example 1. The above-described liquid rubber and the above-described filler are uniformly mixed with each other at the above-described formulation ratio. A molding material was prepared in this manner. Then, the molding material was poured into molding dies for the evaluation samples and cured.

An acoustic impedance evaluation sample A and a tear strength evaluation sample B were produced as the evaluation samples of the Example 1.

The molding material was molded into a sheet shape of 30 mm×30 mm×1 mm as the sample A. Heating and curing for 1 hour at 100° C. was used as the curing method.

The molding material was molded into a shape of a test piece in accordance with JIS K 6252 as the sample B. Specifically, an uncut angle-type test piece having a thickness of 0.5 mm was produced. Heating and curing for 1 hour at 100° C. was used as the curing method.

### Example 2

In the resin composition of Example 2, 55 parts by mass of silicone rubber was used as the elastomer substrate 31 and 45 parts by mass of a crystalline filler made of zinc oxide was used as the filler 32.

The material of the silicone rubber and the curing agent were the same as those in Example 1.

The average maximum length of the filler 32 was 10 μm, and the average maximum diameter thereof was 0.2 μm.

The same samples A and B as those in Example 1 were produced as evaluation samples of Example 2 in the same manner as in Example 1 except that a different molding material was used.

### Example 3

Example 3 is a modification example of the above-described first embodiment.

In the resin composition of Example 3, the same configuration as that of Example 1 was used except that the content of the filler 32 was set to 30 parts by mass and 5 parts by mass of alumina (with an average particle diameter of 0.03 μm) was added as the filler 33.

The same samples A and B as those in Example 1 were produced as evaluation samples of Example 3 in the same manner as in Example 1 except that a different molding material was used.

### Example 4

Example 4 is a modification example of the above-described first embodiment.

In the resin composition of Example 4, 60 parts by mass of silicone rubber was used as the elastomer substrate 31, 30 parts by mass of a crystalline filler made of magnesium sulfate was used as the filler 32, and 10 parts by mass of zirconia (with an average particle diameter of 0.1 μm) was used as the filler 33.

The material of the silicone rubber and the curing agent were the same as those in Example 1.

The average maximum length of the filler 32 was 15 μm, and the average maximum diameter thereof was 0.5 μm.

The same samples A and B as those in Example 1 were produced as evaluation samples of Example 4 in the same manner as in Example 1 except that a different molding material was used.

### Example 5

In the resin composition of Example 5, the same configuration as that of Example 1 was used except that 30 parts by mass of a crystalline filler made of potassium titanate and 5 parts by mass of a glass filler were used as the filler 32. The same material as that of Example 1 was used as the crystalline filler made of potassium titanate.

The average maximum length of the glass filler was 100 μm, and the average maximum diameter thereof was 1 μm.

The same samples A and B as those in Example 1 were produced as evaluation samples of Example 5 in the same manner as in Example 1 except that a different molding material was used.

### Comparative Example

In the resin composition of the comparative example, the same configuration as that of Example 1 was used except that the 35 parts by mass of alumina (with an average particle diameter of 1 μm) was used instead of the filler 32.

The same samples A and B as those in Example 1 were produced as evaluation samples of the comparative example in the same manner as in Example 1 except that a different molding material was used.

### Evaluation

The acoustic impedance was obtained using the samples A in each of the examples and the comparative example. First, the density and the acoustic velocity of each of the resin compositions of the samples A in each of the examples and the comparative example were measured. The acoustic impedance was calculated by the product of the measured density and acoustic velocity.

The tear strength was measured by carrying out a tear strength test in accordance with JIS K 6252 using the samples B in each of the examples and the comparative example.

### Evaluation Result

As shown in [Table 1], the acoustic impedance of the samples A in Examples 1 and 3 to 5 was 1.30 MRay1, and the acoustic impedance in Example 2 was 1.40 MRay1.

The acoustic impedance of the sample A of the comparative example was 1.31 MRay1.

The acoustic impedance of an acoustic lens is suitably 1.3 MRay1 to 1.6 MRay1 which is close to the acoustic impedance of biological tissue. All of the examples and the comparative example had acoustic impedance suitable as the acoustic lens 30.

As shown in [Table 1], the tear strengths of the samples B in Examples 1 to 5 were respectively 43 N/mm, 48 N/mm, 41 N/mm, 41 N/mm, and 49 N/mm.

The tear strength of the sample B in the comparative example was 11 N/mm.

On the other hand, the tear strength of a cured product of a silicone rubber alone used in each resin composition was 28 N/mm. The tear strengths of the samples B in Examples 1 to 5 were 1.46 to 1.75 times the tear strength of the silicone alone, and as a result, the tear strengths of the samples B in Examples 1 to 5 were excellent.

In particular, both the acoustic impedance and the tear strength in Example 2 were larger than those in Example 1 due to a higher content of the filler than that of Example 1.

Although the content of the filler in Examples 3 and 4 was smaller than that of Example 1, the samples were supplemented with inorganic particles instead of the reduced amount of filler. As a result, there was no change in acoustic impedance compared to Example 1. However, the tear strengths in Examples 3 and 4 were slightly reduced compared to that of Example 1.

Example 5 is an example in which a plurality of filler materials are mixed as the filler 32.

Part of the filler made of potassium titanate in Example 1 was replaced with a glass filler in Example 5. In this case, there was no change in acoustic impedance even if the glass filler was added. However, as a result of adding the glass filler having larger average maximum length and average maximum diameter, the tear strength was higher than that of Example 1.

The tear strength of the comparative example was reduced to 0.39 times the tear strength of the silicone alone by incorporating inorganic particles.

It is considered that, in a case where the content of the inorganic particles in the comparative example is reduced, the tear strength of the comparative example approaches the strength of the silicone alone. However, even in a case where the content of the inorganic particles is set to 0 parts by mass in the comparative example, the tear strength of the comparative example is significantly lower than the tear strength in each example.

Furthermore, in a case where the content of the inorganic particles in the comparative example is reduced, it is clear that the acoustic impedance is reduced below 1.31. Accordingly, there is a high possibility that the acoustic impedance is not favorable as an acoustic lens.

While preferred embodiments of the invention have been described and illustrated above, it should be understood that these are exemplary of the invention and are not to be considered as limiting. Additions, omissions, substitutions, and other modifications can be made without departing from the spirit or scope of the present invention.

Accordingly, the invention is not to be considered as being limited by the foregoing description, and is only limited by the scope of the appended claims.

According to each of the embodiments, it is possible to provide an acoustic lens, an ultrasonic probe, and an ultrasonic diagnostic device which have favorable acoustic characteristics and excellent mechanical durability.

What is claimed is:

1. An acoustic lens comprising:
an elastomer substrate; and
a filler which is dispersed in the elastomer substrate and has an average maximum length of 5 μm to 100 μm and an average maximum diameter of 0.01 μm to 1 μm.

2. The acoustic lens according to claim 1,
wherein the filler includes a crystalline inorganic filler.

3. The acoustic lens according to claim 1,
wherein a material of the filler includes at least one selected from a group consisting of potassium titanate, zinc oxide, and magnesium sulfate.

4. An ultrasonic probe comprising:
an acoustic lens including:
an elastomer substrate; and
a filler which is dispersed in the elastomer substrate and has an average maximum length of 5 μm to 100 μm and an average maximum diameter of 0.01 μm to 1 μm.

5. An ultrasonic diagnostic device comprising:
an acoustic lens including:
an elastomer substrate; and
a filler which is dispersed in the elastomer substrate and has an average maximum length of 5 μm to 100 μm and an average maximum diameter of 0.01 μm to 1 μm.
